(19) United States
(12) Patent Application Publication
(10) Pub. No.: US 2025/0073367 A1
(43) Pub. Date: Mar. 6, 2025

(54) COMPOSITIONS PROVIDING AN EFFECTIVE MICROBIAL INHIBITORY CONCENTRATIONS OF ANTIMICROBIAL COMPOUNDS

(71) Applicant: S.C. Johnson & Son, Inc., Racine, WI (US)

(72) Inventor: Michael S. Haas, Franklin, WI (US)

(73) Assignee: S.C. Johnson & Son, Inc., Racine, WI (US)

(21) Appl. No.: 18/824,307

(22) Filed: Sep. 4, 2024

Related U.S. Application Data
(60) Provisional application No. 63/536,773, filed on Sep. 6, 2023.

(51) Int. Cl. A61L 9/12; A61L 101/34; A61L 101/40

(52) U.S. Cl. CPC A61L 9/127; A61L 2101/34; A61L 2101/40; A61L 2209/131; A61L 2209/135

(57) ABSTRACT

The present disclosure relates to compositions and associated apparatuses that provide an effective microbial inhibitory headspace concentration of allyl isothiocyanate, citral, thymol, linalool, terpincol, eugenol, or a combination of any two or more thereof.

### CROSS-REFERENCE TO RELATED PATENT APPLICATIONS

This application claims the benefit of and priority to U.S. Provisional Appl. No. 63/536,773, filed Sep. 6, 2023, the contents of which are incorporated herein by reference in their entirety for any and all purposes.

### FIELD

The present technology is directed to compositions and associated apparatuses that provide an effective microbial inhibitory headspace concentration of allyl isothiocyanate, citral, thymol, linalool, terpineol, eugenol, or a combination of any two or more thereof.

### SUMMARY

In an aspect of the present technology, a composition is disclosed for providing an effective microbial inhibitory headspace concentration of allyl isothiocyanate, citral, thymol, linalool, terpineol, eugenol, or a combination of any two or more thereof, wherein the composition includes allyl isothiocyanate, citral, thymol, linalool, terpineol, eugenol, or a combination of any two or more thereof; a fragrance; and optionally an emulsifier. As a person of ordinary skill in the art would understand from the present disclosure and readily perform based on the present disclosure, a particular effective microbial inhibitory headspace concentration for a particular component (i.e., allyl isothiocyanate, citral, thymol, linalool, terpincol, eugenol, or a combination of any two or more thereof) may be provided by adjusting the amount of the component(s) in the composition given variables such as (i) room size, (ii) the type of delivery device, (iii) the emission rate of the delivery device, (iv) the reservoir size of the delivery device, etc.

In an aspect of the present technology, an apparatus is provided that includes a composition according to any embodiment disclosed herein wherein the apparatus is configured to provide an effective microbial inhibitory headspace concentration of allyl isothiocyanate, citral, thymol, linalool, terpineol, eugenol, or a combination of any two or more thereof. The apparatus may be, e.g., an air freshening apparatus, and may be configured for use in a variety of applications to deliver the allyl isothiocyanate, citral, thymol, linalool, terpineol, eugenol, or combination of any two or more thereof to the atmosphere.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, p³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SFs), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; and nitriles (i.e., CN).

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi-or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi-and tricyclic ring systems include both bridged cycloalkyl groups and fused rings, such as, but not limited to, bicyclo[2.1.1] hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl,-CH=CH (CH3), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di-or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

The term “alkenal” refers to a compound including at least one alkenyl group and at least one aldehyde group.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, and —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted.

Heterocyclylalkyl groups are alkyl groups as defined above in which a hydrogen or carbon bond of an alkyl group is replaced with a bond to a heterocyclyl group as defined above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “carboxylate” as used herein refers to a —COOH group.

The term “ester” as used herein refers to —COOR⁷⁰ and —C(O)O—G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “imine” refers to —CR¹⁰⁰(NR¹⁰¹) and —N(CR¹⁰⁰R¹⁰¹) groups, wherein R¹⁰⁰ and R¹⁰¹ are cach independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

It will be understood that phrase “Cₓ-C_{y}” such as “C₅-C₈ alkenal”, means the indicated compound has a total number of carbon atoms falling in the range from x to y. The phrase “C_{z}+” or “C_{z} plus” will be understood to include compounds with a carbon number of z or greater; likewise, the phrase “C_{w}−” or “C_{w} minus” will be understood to include compounds with a carbon number of w or less.

As understood by one of ordinary skill in the art, “molecular weight” (also known as “relative molar mass”) is a dimensionless quantity but is converted to molar mass by multiplying by 1 gram/mole or by multiplying by 1 Da—for example, a compound with a weight-average molecular weight of 5,000 has a weight-average molar mass of 5,000 g/mol and a weight-average molar mass of 5,000 Da.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Throughout this disclosure, various publications, patents and published patent specifications are referenced by an identifying citation. Also within this disclosure are Arabic numerals referring to referenced citations, the full bibliographic details of which are provided subsequent to the Examples section. The disclosures of these publications, patents and published patent specifications are hereby incorporated by reference into the present disclosure to more fully describe the present technology.

As used herein, the term “interior space” refers to a finite volume of space in a residential, commercial or vehicle environment.

As used herein, the term “interior surfaces” refers to surfaces of objects in an interior space. Such objects may include but is not limited to, walls, ceilings, floors, wall dividers, windows, doors, trim, area rugs, carpeting, wall, hangings, vents, beds, chairs, toilets, refrigerators, kitchen cabinets, sinks, trash cans, curtains, towels, clothes, car seats, sofas, furniture, or the like.

As used herein, the term “membrane” refers to a semi-permeable material which allows some components of matter to pass through but stops other components. Of the components that pass through, the membrane moderates the permeation of components i.e. some components permeate faster than other components. Such components may include molecules, ions or particles.

As used herein, the term “permeable material” refers to any material that allows liquids or gases to pass through, and includes, but is not limited to, drywall, wallpaper, wood, vinyl, plastic, plaster, wallboard, fabrics, upholstery, paper, wovens, natural polymers, synthetic polymers and inorganic materials and mixtures thereof. The permeable material may also include residue formed on a surface.

As used herein, the term “vaporize” or “vaporization” refers to a phase transition of a substance or a compound from a solid and/or liquid phase to vapor phase.

### The Present Technology

Microbial growth remains a nagging issue in many interior spaces used by people, and especially in hot & humid regions. Bleach and other disinfectant products are harsh when applied and only kills microbes at the time of use, where any microbes not killed quickly return to a perceivable level. In addition, there is a strong consumer desire for products able to inhibit microbial growth and resulting stains and odors.

The present technology responds to these needs as well as provides additional advantages, providing compositions and apparatuses allowing for continuous to intermittent—continuous release of particular antimicrobial compounds. The present technology further allows for such release to occur with an associated pleasing fragrance.

Thus, in an aspect, the present technology includes a composition for providing an effective microbial inhibitory headspace concentration of allyl isothiocyanate, citral, thymol, linalool, terpineol, eugenol, or a combination of any two or more thereof, wherein the composition includes allyl isothiocyanate, citral, thymol, linalool, terpineol, eugenol, or a combination of any two or more thereof; a fragrance; and optionally an emulsifier. For case of reference, the compositions included in any aspect or embodiment herein may be referred to anywhere in this disclosure as “a composition of the present technology,” “compositions of the present technology,” or the like.

An “effective microbial inhibitory headspace concentration” refers to a vapor concentration of a compound in a gas where the vapor concentration of the compound in the gas, as measured when the gas is at about 35° C. and at a relative humidity of about 80%, is effective to inhibit the growth of one or more microbes on an interior surface. Such microbes may be Aspergillus niger, Pseudomonas aeruginosa, and/or Staphylococcus aureus. The particular “headspace” containing the gas is an interior space, such as a small room or small space. Small rooms may include, but are not limited to, a bathroom, a toilet, a locker room, or the like. Small spaces may include, but are not limited to, a storage spaces for garbage, a toilet bowl, a closet, furniture for storage of shoes, a pet litter box, a pet carrier, a gym locker, or the like. Pet carriers are small portable boxes, crates, and/or cages used to transport small animals such as cats, dogs, hamsters, guinea pigs, or the like. The headspace volume in any aspect or embodiment herein may be from about 7 liters to about 5660 Liters. In any aspect or embodiment disclosed herein, the effective microbial inhibitory headspace concentration may be about 1×10⁻⁵ μg/L to about 10 ug/L of allyl isothiocyanate, citral, thymol, linalool, terpineol, eugenol, or a combination of any two or more thereof. Thus, in any aspect or embodiment disclosed herein, the effective microbial inhibitory headspace concentration of allyl isothiocyanate, citral, thymol, linalool, terpineol, eugenol, or a combination of any two or more thereof may be about 1×10⁻⁵ μg/L, about 2×10⁻⁵ μg/L, about 3×10⁻⁵ μg/L, about 4×10⁻⁵ μg/L, about 5×10⁻⁵ μg/L, about 6×10⁻⁵ μg/L, about 7×10⁻⁵ μg/L, about 8×10⁻⁵ μg/L, about 9×10⁻⁵ μg/L, about 1×10⁻⁴ μg/L, about 2×10⁻⁴ μg/L, about 3×10⁻⁴ μg/L, about 4×10⁻⁴ μg/L, about 5×10⁻⁴ μg/L, about 6×10⁻⁴ μg/L, about 7×10⁻⁴ μg/L, about 8×10⁻⁴ μg/L, about 9×10⁻⁴ μg/L, about 1×10⁻³ μg/L, about 2×10⁻³ μg/L, about 3×10⁻³ μg/L, about 4×10⁻³ μg/L, about 5×10⁻³ μg/L, about 6×10⁻³ μg/L, about 7×10⁻³ μg/L, about 8×10⁻³ μg/L, about 9×10⁻³ μg/L, about 1×10⁻² μg/L, about 2×10⁻² μg/L, about 3×10⁻² μg/L, about 4×10⁻² μg/L, about 5×10⁻² μg/L, about 6×10⁻² μg/L, about 7×10⁻² μg/L, about 8×10⁻² μg/L, about 9×10⁻² μg/L, about 0.1 μg/L, about 0.2 μg/L, about 0.3 μg/L, about 0.4 μg/L, about 0.5 μg/L, about 0.6 μg/L, about 0.7 μg/L, about 0.8 μg/L, about 0.9 μg/L, about 1 μg/L, about 2 μg/L, about 3 μg/L, about 4 μg/L, about 5 μg/L, about 6 μg/L, about 7 μg/L, about 8 μg/L, about 9 μg/L, about 10 μg/L, or any range including and/or in-between any two of these values. As a person of ordinary skill in the art would understand from the present disclosure and readily perform in view of the present disclosure, a particular effective microbial inhibitory headspace concentration for a particular component (i.e., allyl isothiocyanate, citral, thymol, linalool, terpincol, eugenol, or a combination of any two or more thereof) may be provided by adjusting the amount of the component(s) in the composition given variables such as (i) room size, (ii) the type of delivery device, (iii) the emission rate of the delivery device, (iv) the reservoir size of the delivery device, etc.

Without being bound by theory, it is believed that the effective microbial inhibitory headspace concentration of allyl isothiocyanate, citral, thymol, linalool, terpincol, eugenol, or a combination of any two or more thereof provided by the present technology results in disruption of microbial enzymes, disruption of microbial metabolic function (e.g., by diffusing through the cell wall), and/or disruption of the polysaccharide integrity of microbial cell wall.

In any embodiment herein, a composition of the present technology may include allyl isothiocyanate, citral, thymol, linalool, terpineol, eugenol, or a combination of any two or more thereof in an amount of about 0.25 wt. % to about 25 wt. % (based on the total weight of the composition). Thus, in any embodiment herein, a composition of the present technology may include allyl isothiocyanate, citral, thymol, linalool, terpineol, eugenol, or a combination of any two or more thereof in an amount of about 0.25 wt. %, about 0.30 wt. %, about 0.35 wt. %, about 0.40 wt. %, about 0.45 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, about 10 wt. %, about 10.5 wt. %, about 11 wt %, about 11.5 wt. %, about 12 wt. %, about 12.5 wt. %, about 13 wt. %, about 13.5 wt. %, about 14 wt. %, about 14.5 wt %, about 15 wt %, about 15.5 wt %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, or any range including and/or in-between any two of these values.

The composition may, in any embodiment herein, include allyl isothiocyanate and optionally be substantially free of one or more of citral, thymol, linalool, terpineol, or eugenol. The phrase “substantially free” as used in this disclosure will be understood to mean that the composition includes less than 0.01 wt. % of the indicated component(s). For example, a composition of the present technology may include allyl isothiocyanate and be substantially free of citral. Further, a composition of the present technology may include allyl isothiocyanate and not include one or more of citral, thymol, linalool, terpineol, or eugenol. Thus, a composition of the present technology may include allyl isothiocyanate and not include citral.

A composition of the present technology may, in any embodiment herein, be substantially free of a mixture of (a) an unbranched unsubstituted linear C₅-C₈ alkenal, and (b) an unbranched unsubstituted linear C₉-C₁₄ alkenal. Further, in any embodiment herein it may be that a composition of the present technology does not include a mixture of (a) an unbranched unsubstituted linear C₅-C₈ alkenal, and (b) an unbranched unsubstituted linear C₉-C₁₄ alkenal.

In any embodiment herein, the composition may include about 0.25 wt. % to about 3.0 wt. % of allyl isothiocyanate. Thus, in any embodiment herein, a composition of the present technology may include allyl isothiocyanate in an amount of about 0.25 wt. %, about 0.30 wt. %, about 0.35 wt. %, about 0.40 wt. %, about 0.45 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, or any range including and/or in-between any two of these values.

In any embodiment herein, the composition may include about 0.25 wt. % to about 8.1 wt. % of citral; accordingly, in any embodiment herein a composition of the present technology may include citral in an amount of about 0.25 wt. %, about 0.30 wt. %, about 0.35 wt. %, about 0.40 wt. %, about 0.45 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, or any range including and/or in-between any two of these values.

The composition may, in any embodiment herein of the present technology, include about 0.25 wt. % to about 3.0 wt. % of thymol. Accordingly, in any embodiment herein a composition of the present technology may include thymol in an amount of about 0.25 wt. %, about 0.30 wt. %, about 0.35 wt. %, about 0.40 wt. %, about 0.45 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, or any range including and/or in-between any two of these values.

The composition may, in any embodiment herein of the present technology, include about 0.25 wt. % to about 20 wt. % of linalool. Thus, a composition of the present technology may, in any embodiment herein, include linalool in an amount of about 0.25 wt. %, about 0.30 wt. %, about 0.35 wt. %, about 0.40 wt. %, about 0.45 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, about 10 wt. %, about 10.5 wt. %, about 11 wt %, about 11.5 wt. %, about 12 wt. %, about 12.5 wt. %, about 13 wt. %, about 13.5 wt. %, about 14 wt. %, about 14.5 wt %, about 15 wt %, about 15.5 wt %, about 16 wt. %, about 17 wt. %, about 18 wt. %, about 19 wt. %, about 20 wt. %, or any range including and/or in-between any two of these values.

In any embodiment herein, the composition may include about 0.25 wt. % to about 20 wt. % of terpineol. Thus, a composition of the present technology may, in any embodiment herein, include terpineol in an amount of about 0.25 wt. %, about 0.30 wt. %, about 0.35 wt. %, about 0.40 wt. %, about 0.45 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, about 10 wt. %, about 10.5 wt. %, about 11 wt %, about 11.5 wt. %, about 12 wt. %, about 12.5 wt. %, about 13 wt. %, about 13.5 wt. %, about 14 wt. %, about 14.5 wt %, about 15 wt %, about 15.5 wt %, about 16 wt. %, about 17 wt. %, about 18 wt. %, about 19 wt. %, about 20 wt. %, or any range including and/or in-between any two of these values.

In any embodiment herein, the composition may include about 0.25 wt. % to about 25 wt. % of eugenol. Thus, a composition of the present technology may, in any embodiment herein, include eugenol in an amount of about 0.25 wt. %, about 0.30 wt. %, about 0.35 wt. %, about 0.40 wt. %, about 0.45 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, about 10 wt. %, about 10.5 wt. %, about 11 wt %, about 11.5 wt. %, about 12 wt. %, about 12.5 wt. %, about 13 wt. %, about 13.5 wt. %, about 14 wt. %, about 14.5 wt %, about 15 wt %, about 15.5 wt %, about 16 wt. %, about 17 wt. %, about 18 wt. %, about 19 wt. %, about 20 wt. %, or any range including and/or in-between any two of these values.

As disclosed above, compositions of the present technology include a fragrance and may optionally include an emulsifier. Suitable fragrances include, but are not limited to, an odor masking agent, an odor blocking agent, and/or a perfume raw material. “Odor blocking” refers to the ability of a compound to dull the human sense of smell. “Odor-masking” refers to the ability of a compound to mask or hide a smell of another compound. Odor-masking may include a compound with a non-offensive or pleasant smell that is dosed such it limits the ability to sense the scent of another compound. Odor-masking may involve the selection of compounds which coordinate with an anticipated undesired scent to change the perception of the overall scent provided by the combination of compounds. The fragrance in any embodiment herein may or may not include allyl isothiocyanate, citral, thymol, linalool, terpincol, eugenol, or a combination of any two or more thereof. Suitable emulsifiers include, but are not limited to, polyvinylpyrrolidone (“PVP”), sorbitan trioleate, oleic acid, citric acid, a polyoxyethylene lauryl ether, and/or a polyoxyethylene sorbitan monolaurate (e.g., polyoxyethylene (20) sorbitan monolaurate). The emulsifier may be included in an amount of about 0.01 wt. % to about 1.0 wt. % of the composition; thus, in any embodiment herein, the composition may include the emulsifier at about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.10 wt. %, about 0.15 wt. %, about 0.20 wt. %, about 0.25 wt. %, about 0.30 wt. %, about 0.35 wt. %, about 0.40 wt. %, about 0.45 wt. %, about 0.5 wt. %, about 0.6 wt. %, about 0.7 wt. %, about 0.8 wt. %, about 0.9 wt. %, about 1.0 wt. %, or any range including and/or in between any two of these values.

The composition according to any embodiment herein may further include a thickening agent, a foaming agent, and/or a wetting agent. The thickening agent may, in any embodiment herein, include carboxymethyl cellulose, sodium alginate, carrageenan, and/or zanthan gum. The foaming agent may, in any embodiment herein, include a sucrose fatty acid esters, a maltose fatty acid ester, and/or sodium lauroyl sarcosinate. The wetting agent may, in any embodiment herein, include glycerol, sorbitol, and/or propylene glycol.

The composition according to any embodiment herein may further include a diluent. Exemplary diluents include, but are not limited to, dipropylene glycol methyl ether and/or 3-methoxy-3-methyl-1-butanol.

In an aspect of the present technology, an apparatus is provided that includes a composition according to any embodiment disclosed herein wherein the apparatus is configured to provide an effective microbial inhibitory headspace concentration of allyl isothiocyanate, citral, thymol, linalool, terpineol, eugenol, or a combination of any two or more thereof. In any embodiment disclosed herein, the apparatus may be configured to provide an effective microbial inhibitory headspace concentration of about 1×10⁻⁵ μg/L to about 10 μg/L of allyl isothiocyanate, citral, thymol, linalool, terpineol, eugenol, or a combination of any two or more thereof; thus, in any embodiment disclosed herein, the apparatus may be configured to provide an effective microbial inhibitory headspace concentration of allyl isothiocyanate, citral, thymol, linalool, terpineol, eugenol, or a combination of any two or more thereof of about 1×10⁻⁵ μg/L, about 2×10⁻⁵ μg/L, about 3×10⁻⁵ μg/L, about 4×10⁻⁵ μg/L, about 5×10⁻⁵ μg/L, about 6×10⁻⁵ μg/L, about 7×10⁻⁵ μg/L, about 8×10⁻⁵ μg/L, about 9×10⁻⁵ μg/L, about 1×10⁻⁴ μg/L, about 2×10⁻⁴ μg/L, about 3×10⁻⁴ μg/L, about 4×10⁻⁴ μg/L, about 5×10⁻⁴ μg/L, about 6×10⁻⁴ μg/L, about 7×10⁻⁴ μg/L, about 8×10⁻⁴ μg/L, about 9×10⁻⁴ μg/L, about 1×10⁻³ μg/L, about 2×10⁻³ μg/L, about 3×10⁻³ μg/L, about 4×10⁻³ μg/L, about 5×10⁻³ μg/L, about 6×10⁻³ μg/L, about 7×10⁻³ μg/L, about 8×10⁻³ μg/L, about 9×10⁻³ μg/L, about 1×10⁻² μg/L, about 2×10⁻² μg/L, about 3×10⁻² μg/L, about 4×10⁻² μg/L, about 5×10⁻² μg/L, about 6×10⁻² μg/L, about 7×10⁻² μg/L, about 8×10⁻² μg/L, about 9×10⁻² μg/L, about 0.1 μg/L, about 0.2 μg/L, about 0.3 μg/L, about 0.4 μg/L, about 0.5 μg/L, about 0.6 μg/L, about 0.7 μg/L, about 0.8 μg/L, about 0.9 μg/L, about 1 μg/L, about 2 μg/L, about 3 μg/L, about 4 μg/L, about 5 μg/L, about 6 μg/L, about 7 μg/L, about 8 μg/L, about 9 μg/L, about 10 μg/L, or any range including and/or in-between any two of these values. The apparatus may be, e.g., an air freshening apparatus, and may be configured for use in a variety of applications to deliver the allyl isothiocyanate, citral, thymol, linalool, terpincol, eugenol, or combination of any two or more thereof to the atmosphere. The apparatus in any embodiment herein may be non-energized (where the apparatus is passive and does not require powering by a source of external energy such as heat, gas, or electrical current) or energized. An exemplary energized apparatus according to the present technology may be an electrical device, such as an electrical wall plug or battery operated air freshener having a wick and/or a membrane as described herein to transport the allyl isothiocyanate, citral, thymol, linalool, terpineol, and/or eugenol of the composition and/or vaporize the allyl isothiocyanate, citral, thymol, linalool, terpineol, and/or eugenol of the composition.

The apparatus may, in any embodiment herein, include a reservoir for the composition. The apparatus may include a delivery member configured to contain a liquid phase of the composition and allow the liquid phase of the composition to vaporize therefrom. Suitable delivery members include, but are not limited to, a wick, a breathable membrane (e.g., a permeable membrane and/or a semi-permeable membrane), a gel, a porous substrate, and/or a semi-porous substrate optionally including a felt pad. For example, the delivery member of any embodiment of the present technology may be a semi-permeable membrane. In any embodiment herein including a reservoir for the composition, the apparatus may include a rupturable substrate scalably attached to and covering the reservoir to prevent the release of any component of the composition until the apparatus is activated. The rupturable substrate may be ruptured to release the the allyl isothiocyanate, citral, thymol, linalool, terpincol, and/or eugenol of the composition by actuating a rupture mechanism positioned adjacent to the rupturable substrate.

In any embodiment herein including a reservoir for the composition, the apparatus may include the reservoir as part of a container. The container may be constructed from a material that prevents diffusion of a vapor phase of the composition from the apparatus prior to use, where examples of such materials include metal, glass, ceramic, and/or plastic. The container may, in any embodiment herein, contain about 1 mL to about 50 mL of the composition. The container may include a membrane disposed adjacent to the reservoir to provide for a vapor phase of the composition to pass through, where the membrane may be a microporous membrane exhibiting an average pore size of about 0.01 microns to about 0.06 microns; thus, the average pore size may be about 0.01 microns, about 0.02 microns, about 0.03 microns, about 0.04 microns, about 0.05 microns, about 0.06 microns, or any range including and/or in between any two of these values.

In any embodiment herein, the membrane may be filled with a suitable filler and/or plasticizer as understood by a person of ordinary skill in the art (e.g., the microporous membrane may include about 50 wt. % to about 80 wt. % silica). Suitable microporous membranes include, but are not limited to, a microporous, ultra-high molecular weight polyethylene optionally filled with silica as described in U.S. Pat. No. 7,498,369. In any embodiment herein, the membrane (e.g., microporous membrane) may have a thickness of about 0.01 millimeter to about 1 millimeter. Thus, in any embodiment herein, the membrane (e.g., microporous membrane) may have a thickness of about 0.01 millimeter, about 0.02 millimeters, about 0.03 millimeters, about 0.04 millimeters, about 0.05 millimeters, about 0.06 millimeters, about 0.07 millimeters, about 0.08 millimeters, about 0.09 millimeters, about 0.10 millimeters, about 0.11 millimeters, about 0.12 millimeters, about 0.13 millimeters, about 0.14 millimeters, about 0.15 millimeters, about 0.16 millimeters, about 0.17 millimeters, about 0.18 millimeters, about 0.19 millimeters, about 0.20 millimeters, about 0.30 millimeters, about 0.40 millimeters, about 0.50 millimeters, about 0.60 millimeters, about 0.70 millimeters, about 0.80 millimeters, about 0.90 millimeters, about 1 millimeter, or any range including and/or in between any two of these values. In any embodiment herein, the membrane (e.g., microporous membrane) may include an evaporative surface area of about 2 cm² to about 100 cm². Thus, in any embodiment herein, the evaporative surface area of the membrane (e.g., microporous membrane) may be about 2 cm², about 3 cm², about 4 cm², about 5 cm², about 6 cm², about 7 cm², about 8 cm², about 9 cm², about 10 cm², about 15 cm², about 20 cm², about 25 cm², about 30 cm², about 35 cm², about 40 cm², about 45 cm², about 50 cm², about 60 cm², about 70 cm², about 80 cm², about 90 cm², about 100 cm², or any range including and/or in between any two of these values.

In any embodiment herein of the apparatus that includes a wick, the wick may be composed of various materials and construction, including, but not limited to, bundled fibers compressed and/or formed into various shapes via overwrap (e.g., a non-woven sheet over-wrap) or sintered plastics such as polyethylene. The wick may be configured to have various different shapes and sizes (e.g., a cylindrical or an elongate cube shape). The wick may be defined by a length and a diameter or width, depending on the shape. In any embodiment, the wick may have a length of about 1 millimeter to about 100 millimeters. In any embodiment, the wick may have diameter or width of about 1 mm or greater. In any embodiment, the wick may have a density of about 0.100 grams/cm³ to about 1 gram/cm³.

The examples herein are provided to illustrate advantages of the present technology and to further assist a person of ordinary skill in the art with preparing or using the compounds and compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects, or embodiments of the present technology.

### EXAMPLES

### Example 1: Model Bathroom Performance of Exemplary Compositions of the Present Technology

Studies were conducted to assess the growth inhibition of Aspergillus niger, Pseudomonas aeruginosa, and Staphylococcus aureus under conditions designed to mimic real bathroom conditions. Tested were (a) a marketed fragrance composition (“Marketed Fragrance 1”), (b) a marketed fragrance composition that included trans-2-hexenal (“Marketed Fragrance 2”), (c) Marketed Fragrance 1 additionally including 1 wt. % mustard essential oil (mustard essential oil containing greater than 95 wt. % allyl isothiocyanate), and (d) Marketed Fragrance 2 additionally including 1 wt. % mustard essential oil (mustard essential oil containing greater than 95 wt. % allyl isothiocyanate). Of these compositions, (a), (c), and (d) were tested against Aspergillus niger, where (a) and (d) were also tested against each of Pseudomonas aeruginosa and Staphylococcus aureus Also tested against Aspergillus niger were (c) Marketed Fragrance 2 additionally including 3 wt. % thymol (as thymol crystals), and (f) Marketed Fragrance 2 additionally including 8 wt. % citral. Of these compositions, (c)-(f) are in accordance with compositions of the present technology. For each composition, 8 grams of cach composition was included in its own test sample cartridge and sealed with metal foil until removed (as indicated below).

For each tested composition with cach of Aspergillus niger, Pseudomonas aeruginosa, and Staphylococcus aureus, two separate 200 ft³ test chambers were constructed with glass separately for toilet and tub room to mimic real conditions. Automatic water flushing facility and automatic temperature and humidity controller were made available. Test rooms and incubator were set at required test conditions. Temperature and relative Humidity inside the room were maintained at 28±2° C. and >80%, respectively.

Preparation of Test Chamber: The chamber walls and floors were washed with a 70% isopropyl alcohol solution and dried. Test rooms were set and conditioned at required test conditions. Temperature and relative Humidity inside each room were maintained as 28±2° C. and >80%, respectively. Automatic water flushing frequency was set as mentioned below:
Bathtub: 2 times/day in the morning only with 1-hour interval (i. Filling the water up to the mark; ii. keeping the water in the tub; iii. draining out—cach approx. 20 minutes). Shower was run four times/day, only in the morning for 5 minutes.
Toilet: 8 times/day (4 times each in the morning and evening with 30 minutes interval for 10 seconds). Wash basin in the toilet will be run 4 times in a day for 30 seconds (morning and evening, 2 times cach).

Glassware: Sterilized glassware was used during the experiment. Suitable flasks or jars for preparing saline solution and spore suspension. The glassware used in the present studies were cleaned with commercial cleaning solution and dried in hot air oven at 60° C. for 3 hours. All nutrients media were autoclaved at 15 psi pressure at 121° C. for 15 minutes.

Culture medium: Potatoes Dextrose Agar (HIMEDIA-M096), at a concentration of 39.0 g/liter potatoes dextrose agar, final pH at 25° C. of 5.6±0.2. Medium was sterilized by autoclaving at 15 psi pressure at 121° C. for 15 minutes, and was mixed well before dispensing.

Preparation of Colloidal Suspension: Sterile potato dextrose agar in plates or flasks were prepared. A. niger, P. aeruginosa, or S. aureus (depending on the study) was inoculated at the center of the agar and incubate for 7-10 days at 28±2° C. in incubator. A sterile water diluent containing 0.85% NaCl and 0.20% Triton X-100 was prepared, and approximately 10 mL of diluent was poured over the surface and agitated vigorously to dislodge the microbes. This solution was poured into a sterilized beaker and subject to ultrasonication for 5 minutes to break up microbial chains. The suspension was filtered through sterile absorbent cotton to remove microbial chains and any hyphal elements. Conidial suspensions was stored at 2-10° C. for a period of 4 weeks for use in preparing test suspensions of conidia. Standardization was done using conidial suspensions as needed by diluting stock spore suspension with saline-surfactant mixture so that it contained (4×10⁶ spores/mL). To prepare test suspension, take 1 mL of the 5 million conidia/mL microbial suspension and placed it into 20 mL sterile Potato Dextrose broth (nutrient solution).

Inoculation and Incubation of Tiles: The paper and glue backing from ceramic tiles were removed and simply washed with deionised water. Dried tiles were sterilized by U.V sterilization and Tiles were inoculated with 50 μL of the microbial suspension (about 9.7×10⁶ spores/mL). Four tiles were used per location by pasting them in an empty petri plate using adherence tape with some distance among them to avoid dispersion of growth. The petri plates with tiles were pasted on the walls with different location. Three sets of tiles were made for incubator, tub room and toilet room. Tiles were kept in a petri plate and placed in incubator with temperature and relative humidity at 35±2° C. and >80%, respectively for 1 hour for drying. After drying, plates were shifted from incubator to conditioned tub room and toilet room except one set were kept in incubator itself. Temperature and relative humidity inside the chambers and incubators were maintained as 28±2° C. and >80%, respectively for 10 days. The metal foil was then removed from the test sample cartridge and placed on a wall of the tub room facing outward for 10 days ageing purpose. Ten tile groups each inoculated with A. niger, P. aeruginosa, or S. aureus placed in plates were adhered to different locations in the rooms no. after 10^{th} day ageing and further kept for another 21 days exposure period with same conditions for comparison.

Observations: Observations for microbial growth were made at the end of the 21 day exposure period.

The results of these studies are provided in Table 1 below, where the “% Inhibition” was determined in relation to the marketed fragrance control; “% Inhibition”=[((Control Inhibition)−(Tested Inhibition))/(Control Inhibition)]×100].

TABLE 1

| | Air Exchange | % Inhibition | % Inhibition | % Inhibition |
|---|---|---|---|---|
| Composition | Rate | A. niger | P. aeruginosa | S. aureus |
| Marketed Fragrance 1 | 0 | 0.00 | [Not Yet Tested] | [Not Yet Tested] |
| Marketed Fragrance 1 + | 0 | 98.38 | [Not Yet Tested] | [Not Yet Tested] |
| 1% Mustard Essential Oi | | | | |
| Marketed Fragrance 2 + | 0 | 96.51 | [Not Yet Tested] | [Not Yet Tested] |
| 1% Mustard Essential Oi | | | | |
| Marketed Fragrance 1 | 0.35 ACH | 0.00 | 0.00 | 0.00 |
| Marketed Fragrance 2 + | 0.35 ACH | 90.89 | 53.14 | 46.61 |
| 1% Mustard Essential Oil | | | | |
| Marketed Fragrance 2 + | 0.35 ACH | 63.03 | [Not Yet Tested] | [Not Yet Tested] |
| 3% Thymol | | | | |
| Marketed Fragrance 2 + | 0.35 ACH | 78.43 | [Not Yet Tested] | [Not Yet Tested] |
| 8% Citral | | | | |

Example 2: 7L Box Inhibition by Exemplary Compositions of the Present Technology

A similar set of experiments was performed as described in Example 1 except, instead of the described 200 ft³ test chamber, 7L containers each made of clear polypropylene were utilized (a “7L Box”). Into each 7L Box was placed petri dishes inoculated with A. niger, P. aeruginosa, or S. aureus, immediately followed by dividing the 7L Boxes into groups into which was placed:
(i) 0.125 grams of Marketed Fragrance 1 as a control;
(ii) 0.125 grams of a composition of Marketed Fragrance 2 additionally including 1.2 wt. % mustard essential oil (mustard essential oil containing greater than 95 wt. % allyl isothiocyanate);
(iii) 0.125 grams of a composition of Marketed Fragrance 2 additionally including 3 wt. % thymol (as thymol crystals);
(iv) 0.125 grams of a composition of Marketed Fragrance 2 additionally including 8 wt. % citral;
(v) 0.125 grams of a composition of Marketed Fragrance 2 additionally including 15 wt. % linalool;
(vi) 0.125 grams of a composition of Marketed Fragrance 2 additionally including 15 wt. % terpineol;
(viii) 0.125 grams of a composition of Marketed Fragrance 2 additionally including 15 wt. % eugenol; or
(ix) nothing additional was placed into the 7L Box;
   whereupon each 7L Box was closed and the microbes in the petri dish allowed to grow for 3 days. Of the above-described compositions, (ii)-(viii) are in accordance with compositions of the present technology. At the end of the 3 day incubation period, observations of the microbial growth were made; Table 2 below provides the lowest percent inhibition for each test group after the 3 day incubation period.

TABLE 2

| | % Inhibition | % Inhibition | % Inhibition |
|---|---|---|---|
| Composition | S. aureus | E. coli | A. niger |
| Marketed Fragrance 1 | 0.00 | 0.00 | 0.00 |
| (Control) | | | |
| Marketed Fragrance 2 + | >99.9 | 96.4 | >99.9 |
| 1.2% Mustard | | | |
| Essential Oil | | | |
| Marketed Fragrance 2 + | 99.9 | 99.0 | >99.9 |
| 3% Thymol | | | |
| Marketed Fragrance 2 + | >99.9 | >99.9 | 99.9 |
| 8% Citral | | | |
| Marketed Fragrance 2 + | >99.9 | 99.8 | 99.8 |
| 15% Linalool | | | |
| Marketed Fragrance 2 + | <40 | 99.2 | >99.9 |
| 15% Terpineol | | | |
| Marketed Fragrance 2 + | <40 | 98.5 | >99.9 |
| 15% Eugenol | | | |

### Example 3: Headspace Concentration Measurements

Another series of experiments (the “7L Box Headspace Concentration” experiments) were performed according to the Example 2 experiments above except (i) inoculated petri dishes were not included, and (ii) at the end of 1 day and 3 day incubation periods the concentration of the compound indicated in Table 3 below—in micrograms per liter (μg/L) of the indicated compound in the air of the box at the end of the 1 day or 3 day incubation period—was determined via a thermal desorption-gas chromatography-mass spectrometry analysis using appropriate calibrations, controls, and protocols. Yet another series of experiments (the “200 ft³ Room Headspace Concentration” experiments) were performed according to the 7L Box Headspace Concentration experiments except utilized the 200 ft³ test chambers discussed in Example 1 and utilized a 4 week incubation period, where Table 3 also includes the gaseous concentration data obtained from these 200 ft³ Room Headspace Concentration experiments.

TABLE 3

| | | 7L Box | | 7L Box | | 200 ft³ Room |
|---|---|---|---|---|---|---|
| | | Measured | | Measured | | Measured |
| | | Gaseous | | Gaseous | | Gaseous |
| | Compound | Concentration | | Concentration | | Concentration |
| Composition | Measured | (1 day) | | (3 day) | | (4 weeks) |
| Marketed Fragrance 2 | trans-2- | 13.577 | μg/L | 3.400 | μg/L | 0.006600 |
| | hexenal | | | | | |
| Marketed Fragrance 2 + | allyl | 1.925 | μg/L | 0.405 | μg/L | 0.003270 |
| 1.2% Mustard | isothiocyanate | | | | | |
| Essential Oil | | | | | | |
| Marketed Fragrance 2 + | thymol | 0.240 | μg/L | 0.196 | μg/L | 0.000200 |
| 3% Thymol | | | | | | |
| Marketed Fragrance 2 + | citral | 0.125 | μg/L | 0.160 | μg/L | 0.002300 |
| 8% Citral | | | | | | |
| Marketed Fragrance 2 + | linalool | 2.461 | μg/L | 2.837 | μg/L | 0.003620 |
| 15% Linalool | | | | | | |
| Marketed Fragrance 2 + | terpineol | 0.091 | μg/L | 0.001 | μg/L | 0.000450 |
| 15% Terpineol | | | | | | |
| Marketed Fragrance 2 + | eugenol | 0.133 | μg/L | 0.002 | μg/L | 0.000970 |

15% Eugenol

As Table 3 illustrates, the percent inhibition and gaseous concentrations yielded by 7L Box experiments provide a data trend representative of the performance of the component in a 200 ft³ room setting. These experiments also further evidence that a particular effective microbial inhibitory headspace concentration for a particular component may readily be provided by adjusting the amount of the component(s) in the composition given variables such as (i) room/space size, (ii) air exchange rate of the room/space, (iii) the type of delivery device, (iv) the emission rate of the delivery device, (v) the reservoir size of the delivery device, etc.

### Example 4: Representative Compositions of the Present Technology

The following compositions of the present technology (Compositions A-F) have been generated and found to exhibit microbial inhibition similar to the compositions presented in Table 1 and/or Table 2.

Composition A.

| Component | Wt. % of Total Composition |
|---|---|
| “Functional Fragrance” | 95.4492 |
| (e.g., Marketed Fragrance 2) | |
| CAB-O-SIL M-5 untreated | 2.2500 |
| fumed silica | |
| Mustard Essential Oil (greater | 1.2000 |
| than 95 wt. % allyl | |
| isothiocyanate) | |
| BHT | 1.0000 |
| Polyoxyethylene (20) | 0.1000 |
| Sorbitan Monolaurate | |
| Dye | 0.0080 |
| Total | 100.00000 |

Composition B.

| Component | Wt. % of Total Composition |
|---|---|
| “Functional Fragrance” | 93.6492 |
| (e.g., Marketed Fragrance 2) | |
| CAB-O-SIL M-5 | 2.2500 |
| Thymol | 3.0000 |
| BHT | 1.0000 |
| Polyoxyethylene (20) | 0.1000 |
| Sorbitan Monolaurate | |
| Dye | 0.0080 |
| Total | 100.00000 |

Composition C.

| Component | Wt. % of Total Composition |
|---|---|
| “Functional Fragrance” | 88.5492 |
| (e.g., Marketed Fragrance 2) | |
| CAB-O-SIL M-5 | 2.2500 |
| Citral | 8.1000 |
| BHT | 1.0000 |
| Polyoxyethylene (20) | 0.1000 |
| Sorbitan Monolaurate | |
| Dye | 0.0080 |
| Total | 100.00000 |

Composition D.

| Component | Wt. % of Total Composition |
|---|---|
| “Functional Fragrance” | 81.6492 |
| (e.g., Marketed Fragrance 2) | |
| CAB-O-SIL M-5 | 2.2500 |
| Linalool | 15.8000 |
| BHT | 1.0000 |
| Polyoxyethylene (20) | 0.1000 |
| Sorbitan Monolaurate | |
| Dye | 0.0080 |
| Total | 100.00000 |

Composition E.

| Component | Wt. % of Total Composition |
|---|---|
| “Functional Fragrance” | 81.6492 |
| (e.g., Marketed Fragrance 2) | |
| CAB-O-SIL M-5 | 2.2500 |
| Terpineol | 15.0000 |
| BHT | 1.0000 |
| Polyoxyethylene (20) | 0.1000 |
| Sorbitan Monolaurate | |
| Dye | 0.0080 |
| Total | 100.00000 |

Composition F.

| Component | Wt. % of Total Composition |
|---|---|
| “Functional Fragrance” | 81.6492 |
| (e.g., Marketed Fragrance 2) | |
| CAB-O-SIL M-5 | 2.2500 |
| Eugenol | 15.0000 |
| BHT | 1.0000 |
| Polyoxyethylene (20) | 0.1000 |
| Sorbitan Monolaurate | |
| Dye | 0.0080 |
| Total | 100.00000 |

While certain embodiments have been illustrated and described. a person with ordinary skill in the art. after reading the foregoing specification, can effect changes. substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:

A. A composition for providing an effective microbial inhibitory headspace concentration of allyl isothiocyanate, citral, thymol, linalool, terpineol, eugenol, or a combination of any two or more thereof, wherein the composition comprises
allyl isothiocyanate, citral, thymol, linalool, terpineol, eugenol, or a combination of any two or more thereof;
a fragrance; and
optionally an emulsifier.

B. The composition of Paragraph A, wherein the effective microbial inhibitory headspace concentration is about 1×10⁻⁵ μg/L to about 10 μg/L of allyl isothiocyanate, citral, thymol, linalool, terpineol, eugenol, or a combination of any two or more thereof.

C. The composition of Paragraph A or Paragraph B, wherein the composition comprises about 0.25 wt. % to about 25 wt. % of allyl isothiocyanate, citral, thymol, linalool, terpineol, eugenol, or a combination of any two or more thereof.

D. The composition of any one of Paragraphs A-C, wherein the composition comprises
about 0.25 wt. % to about 3 wt. % of allyl isothiocyanate,
about 0.25 wt. % to about 8.1 wt. % of citral,
about 0.25 wt. % to about 3 wt. % of thymol,
about 0.25 wt. % to about 20 wt. % of linalool,
about 0.25 wt. % to about 20 wt. % of terpineol,
about 0.25 wt. % to about 25 wt. % of eugenol, or
a combination of any two or more thereof.

E. The composition of any one of Paragraphs A-D, wherein the composition comprises allyl isothiocyanate, thymol, linalool, terpineol, eugenol, or a combination of any two or more thereof.

F. The composition of any one of Paragraphs A-E, wherein the composition comprises allyl isothiocyanate.

G. The composition of any one of Paragraphs A-F, wherein the composition comprises about 1 wt. % to about 1.5 wt. % of allyl isothiocyanate.

H. The composition of any one of Paragraphs A-G, wherein the composition does not comprise citral.

I. An apparatus comprising
a reservoir containing a composition according to any one of Paragraphs A-H;
wherein the apparatus is configured to provide an effective microbial inhibitory headspace concentration of allyl isothiocyanate, citral, thymol, linalool, terpineol, eugenol, or a combination of any two or more thereof.

J. The apparatus of Paragraph I, wherein the apparatus is configured to provide an effective microbial inhibitory headspace concentration of about 1×10⁻⁵ μg/L to about 10 μg/L of allyl isothiocyanate, citral, thymol, linalool, terpineol, eugenol, or a combination of any two or more thereof.

K. The apparatus of Paragraph I or Paragraph J, wherein the apparatus is non-energized.

L. The apparatus of any one of Paragraphs I-K, wherein the apparatus is an air freshening apparatus,

M. The apparatus of any one of Paragraphs I-L, wherein the apparatus comprises a delivery member configured to contain a liquid phase of the composition and allow the liquid phase of the composition to vaporize therefrom.

N. The apparatus of any one of Paragraphs I-M, wherein the apparatus comprises a container, the container comprising the reservoir.

O. The apparatus of any one of Paragraphs I-N, wherein the container comprises a membrane disposed adjacent to the reservoir to provide for a vapor phase of the composition to pass through.

P. The apparatus of Paragraph O, wherein the membrane comprises a microporous membrane exhibiting an average pore size of about 0.01 to about 0.06 microns.

Q. The apparatus of Paragraph O or Paragraph P, wherein the membrane is filled with a suitable filler and/or plasticizer.

R. The apparatus of any one of Paragraphs O-Q, wherein the membrane comprises a thickness of about 0.01 millimeter to about 1 millimeter.

S. The apparatus of any one of Paragraphs O-R, wherein the membrane comprises an evaporative surface area of about 2 cm² to about 100 cm².

T. The apparatus of any one of Paragraphs I-S, wherein the delivery member comprises a wick.

Other embodiments are set forth in the following claims, along with the full scope of equivalents to which such claims are entitled.

What is claimed is:

1. A composition for providing an effective microbial inhibitory headspace concentration of allyl isothiocyanate, citral, thymol, linalool, terpineol, eugenol, or a combination of any two or more thereof, wherein the composition comprises
allyl isothiocyanate, citral, thymol, linalool, terpineol, eugenol, or a combination of any two or more thereof;
a fragrance; and
optionally an emulsifier.

2. The composition of claim 1, wherein the effective microbial inhibitory headspace concentration is about 1×10⁻⁵ μg/L to about 10 μg/L of allyl isothiocyanate, citral, thymol, linalool, terpineol, eugenol, or a combination of any two or more thereof.

3. The composition of claim 1, wherein the composition comprises about 0.25 wt. % to about 25 wt. % of allyl isothiocyanate, citral, thymol, linalool, terpineol, eugenol, or a combination of any two or more thereof.

4. The composition of claim 1, wherein the composition comprises
about 0.25 wt. % to about 3 wt. % of allyl isothiocyanate,
about 0.25 wt. % to about 8.1 wt. % of citral,
about 0.25 wt. % to about 3 wt. % of thymol,
about 0.25 wt. % to about 20 wt. % of linalool,
about 0.25 wt. % to about 20 wt. % of terpineol,
about 0.25 wt. % to about 25 wt. % of eugenol, or
a combination of any two or more thereof.

5. The composition of claim 1, wherein the composition comprises allyl isothiocyanate, thymol, linalool, terpineol, eugenol, or a combination of any two or more thereof.

6. The composition of claim 1, wherein the composition comprises allyl isothiocyanate.

7. The composition of claim 1, wherein the composition comprises about 1 wt. % to about 1.5 wt. % of allyl isothiocyanate.

8. The composition of claim 1, wherein the composition does not comprise citral.

9. An apparatus comprising
a reservoir containing a composition according to claim 1;
wherein the apparatus is configured to provide an effective microbial inhibitory headspace concentration of allyl isothiocyanate, citral, thymol, linalool, terpineol, eugenol, or a combination of any two or more thereof.

10. The apparatus of claim 9, wherein the apparatus is configured to provide an effective microbial inhibitory headspace concentration of about 1×10⁻⁵ μg/L to about 10 μg/L of allyl isothiocyanate, citral, thymol, linalool, terpineol, eugenol, or a combination of any two or more thereof.

11. The apparatus of claim 9, wherein the apparatus is non-energized.

12. The apparatus of claim 9, wherein the apparatus is an air freshening apparatus.

13. The apparatus of claim 9, wherein the apparatus comprises a delivery member configured to contain a liquid phase of the composition and allow the liquid phase of the composition to vaporize therefrom.

14. The apparatus of claim 9, wherein the apparatus comprises a container, the container comprising the reservoir.

15. The apparatus of claim 9, wherein the container comprises a membrane disposed adjacent to the reservoir to provide for a vapor phase of the composition to pass through.

16. The apparatus of claim 15, wherein the membrane comprises a microporous membrane exhibiting an average pore size of about 0.01 to about 0.06 microns.

17. The apparatus of claim 15, wherein the membrane is filled with a suitable filler and/or plasticizer.

18. The apparatus of claim 15, wherein the membrane comprises a thickness of about 0.01 millimeter to about 1 millimeter.

19. The apparatus of claim 15. wherein the membrane comprises an evaporative surface arca of about 2 cm² to about 100 cm².

20. The apparatus of claim 9. wherein the delivery member comprises a wick.
